(19) United States
(12) Patent Application Publication
(10) Pub. No.: US 2008/0219998 A1
(43) Pub. Date: Sep. 11, 2008

(54) Anti-Thrombotic Agents

(76) Inventor: Andras Gruber, Portland, OR (US)

Correspondence Address: STOEL RIVES LLP, 900 SW FIFTH AVENUE, SUITE 2600, PORTLAND, OR, 97204-1268, US

(21) Appl. No.: 10/486,159

(22) PCT Filed: May 8, 2002
(86) PCT No.: PCT/US02/14510

§ 371 Date: Dec. 3, 2007

(30) Foreign Application Priority Data
Dec. 7, 2001 (US) 60340294

(51) Int. Cl. A61K 39/395; A61K 38/02; A61K 31/7052; A61K 38/48; A61K 38/10; C07K 16/00; C07K 2/00; C07H 21/00; C12N 9/48; C12N 9/74; C07K 7/08; G01N 33/68; A61P 7/02

(52) U.S. Cl. 424/158.1; 514/2; 514/44; 424/94.64; 514/14; 530/387.1; 530/300; 536/23.1; 435/212; 435/214; 530/327; 436/86

(57) ABSTRACT

The present invention embodies: methods; compounds, their pharmaceutically acceptable analogs, isomer, salts, hydrates, solvates and prodrug derivatives, and pharmaceutically acceptable compositions thereof that have particular biological properties; devices; diagnostic and other assays; and the uses of such methods, compounds, devices and assays. Common throughout these embodiments is specific selective reduction of intravascular thromboplastin antecedent activity, which results in a safe antithrombotic effect. A particularly prominent application or the invention relates to diagnosis and treatment of patients which have, or are at risk of, developing thrombosis, thrombotic injury, or vaso-occlusive diseases, such as myocardial infarction, stroke, restenosis after angioplasty, thrombotic diseases, etc. Another particularly prominent feature of the present invention is its high level of hemostatic safety at optimal efficacy. Also, the present invention is compatible for use in combination with other traditional therapeutic agent such as another antithrombotic, antiplatelet, thrombolytic, or anticoagulant agents.

This invention relates to a new class of pharmaceutical compositions and methods of treatment and prevention of thrombosis and thrombosis related injury and disease. Specifically this invention relates to agents and methods of treatment which prevent thrombosis and thrombosis related diseases without substantially compromising hemostasis More specifically, this invention relates to agents and methods to specifically reduce thrombin-generating thromboplastin antecedent (PTA, coagulation factor XI, FXI) activity in the circulation, including medically useful and pharmaceutically acceptable salts, compositions and dosage forms of compounds, which can provide safe and specific inhibition of thromboplastin antecedent activation, activity, or production, or can enhance the elimination of thromboplastin antecedent, in vivo.

### INTRODUCTION

The circulatory system provides numerous vital functions to the body including, to name a few examples: providing nutrition, providing oxygen, removing CO₂, removing toxic substances and metabolic by products, providing chemical and cellular communication and regulatory agents, and mobilizing defenses against pathogens. Consequently, blood comprises a complex milieu of cellular and molecular components each contributing to the normal functioning state of the individual.

Blood is liquid tissue flowing under pressure and, like all liquids under pressure, to prevent loss must necessarily be maintained in a closed system. That system is the circulatory system comprising the complex of vessels and the heart and providing the necessary interface surfaces to accomplish efficient flow of the blood throughout. When the circulatory structural integrity is breached blood begins to escape. In layman terms when blood is escaping it is referred to as bleeding. The breach must be repaired quickly. This is accomplished by components within the escaping blood itself which instantly react to non-circulatory surfaces and initiate a rapid polymerization event that causes the escaped blood to solidify, thereby “plugging up” the breach and preventing further blood loss. This process is commonly known by laymen as clotting or coagulation and the solidified blood is called a blood clot. If the clotting fails because of a defect in the blood then the bleeding will not stop until the blood pressure has dropped to zero which will surely result in death.

The maintenance of blood in the condition whereby it remains a flowing liquid within the circulatory system is known in the medical profession as homeostasis. The actual mechanism of the blood to coagulate upon contacting external components at the appropriate time and with the appropriate intensity is known as hemostasis. In other words, homeostasis is the normal state of circulating blood characterized by insignificant enzymatic activity of blood coagulation components, such as thrombin generation, and lack of platelet activation, and hemostasis is the coordinated defense mechanism aimed at preserving the integrity of blood circulation upon injury.

Hemostasis includes the coagulation cascade of sequentially activatable enzymes. The cascade is traditionally divided into three parts, an intrinsic pathway, which includes interactions of blood coagulation proteins that lead to the generation of coagulation factor IXa without involvement of coagulation factor VIIa, an extrinsic pathway, which includes interactions of blood coagulation proteins that lead to the generation of coagulation factor Xa and/or IXa without involvement of thromboplastin antecedent (factor XI), and a common coagulation pathway, including interactions of blood coagulation proteins II, V, VIII, IX and X that lead to the generation of thrombin (factor IIa) and, ultimately, fibrin. Coagulation results when fibrinogen is cleaved into fibrin monomers by thrombin and the fibrin monomers in turn form the fibrin polymer.

Once prothrombin (FII) is activated and thrombin (FIIa) is generated in sufficient quantities, coagulation occurs; therefore thrombin is central to hemostasis. Thrombin, activated within the integral circulatory system rather than at the site of injury marks the development of thrombi that can lead to pathological conditions called thrombosis and thrombo-embolism. The layman term for a thrombotic embolus is a floating blood clot that lodges at a site distant from its site of formation. However, as described later, significant compositional, structural and formation differences exist between blood clots and thrombi. The thrombus/thrombi, pathological intravascular tissue-like fibrin/platelet masses that also contains other blood components but not in the same proportion and/or composition as that of blood, can lead to thrombosis, diseases caused by thrombotic blockage of the blood flow to and from organs, and/or thrombin-dependent vaso-occlusive diseases, where thrombin generation plays a pivotal role in the development of vascular occlusions, e.g. coronary thrombosis, restenosis following angioplasty, deep vein thrombosis, disseminated intravascular coagulation in sepsis, etc.

### Improving the Safety of Therapies

Therapeutic modalities are useful only if they are both safe and effective. This invention focuses on improving the safety of antithrombotic therapy. Useful drugs are usually safe. Placebos are useful, very safe, but not specifically efficacious drugs. Many compounds with pharmacodynamic effectiveness in animal models turn out to be unsafe in humans. For example, pain relievers that paralyze the respiratory center of humans at effective doses are useless. The ideal drug is safe at its most efficacious dose. For example, penicillin comes close to being an ideal drug. When administered at its most efficacious doses, penicillin can eradicate infections without jeopardizing the integrity of the host. Unfortunately, many drugs are not safe at their most efficacious doses. Treatment thus often becomes an act of balancing risks and benefits. For example, radiation or chemotherapy can eradicate all forms of cancer but usually at doses that also kill the patient. Most of these modalities thus cannot be used at their most efficacious doses. As a result, cancer remains an important cause of human mortality. Similarly, all anticoagulants currently in use can eradicate thrombosis at doses that cause fatal bleeding complications. These drugs thus cannot be used at their most efficacious doses. As a result, vascular occlusions of thrombotic origin remain the leading cause of human mortality in developed countries.

It is astonishing that typical claims of usefulness of therapeutic compounds are based on efficacy in models. This practice clearly fails to appreciate and comprehend the over thousand year-old yet still valid and solid medical principle that efficacy is a subordinate of safety. Many compounds that are declared useful based on purely efficacy are, in fact, harmful. Relevant claims of usefulness almost exclusively single out effectiveness as a basis for the usefulness claim, which is a clearly deceptive practice. Anticoagulants have always been considered inherently dangerous, thus focusing on efficacy and ignoring the safety of a novel anticoagulant qualifies as malpractice. It is thus not surprising that very few of the hundreds of novel antithrombotic anticoagulants disclosed to date have passed the medical scrutiny of the clinical review board, and even fewer have ever been used or tested in humans. While several inventions recognize the overriding relevance and significance of the safety of an effective therapeutic modality (Jain U.S. Pat. No. 6,214,864; Almstead U.S. Pat. No. 6,218,389; Cupps U.S. Pat. No. 5,804,587; Hensley U.S. Pat. No. 6,346,544; Wright U.S. Pat. No. 5,348,974 each the entirety of which is herein incorporated by reference), only very few exceptions in the anticoagulant field exist. For example, Mertens, U.S. Pat. No. 6,284,871, the entirety of which is herein incorporated by reference, claims usefulness and safety of a monospecific FIX inhibitor as an antithrombotic agent.

### The Relationship Between Blood Coagulation, Hemostasis, and Thrombosis

Referring to FIG. 1, blood is the liquid tissue of circulation. Under normal physiologic conditions of homeostasis, the endothelial barrier efficiently separates blood and other tissues. Diffusion or active transport of proteins and active movement of certain cells across this barrier is not sufficient to produce biologically significant cross-contamination of the two environments that would result in intravascular coagulation and thrombosis.

Referring to FIG. 2, the extrinsic coagulation cascade in the presence of activated platelets initiates both hemostasis and, by expanding into the intravascular space, most cases of thrombosis. Hemostasis following injury starts with activation of the extrinsic coagulation cascade in the presence of activated platelets. The extrinsic coagulation cascade starts with the formation of the tissue factor:factorVII (TFFVII) and then tissue factor:factorVIIa (TFFVIIa) complexes on the surface of tissue factor (TF) expressing cells and extracellular matrix or debris. TFFVIIa then activates both coagulation factors IX and X. More TFFVIIa is converted to TFVIIa by factor Xa (FXa) feedback activation, and the enzyme cascade is accelerated. The FXa:FVa complex then converts prothrombin (FII) into thrombin (FIIa) on membrane surfaces. Thrombin then activates more platelets and, together with factor XIIIa, converts soluble fibrinogen into fibrin polymer that rapidly seals or covers the surface that initiated its own production. Since circulating blood is efficiently sealed off from this “hostile” extracellular environment during hemostasis, and the intravascular antithrombotic system shuts off the positive feed back of continued intravascular thrombin generation, intravascular fibrin generation is normally negligible and self-limiting following transendothelial injury.

Referring to FIG. 3, intravascular progression of the above described thrombin-generating process into thrombosis is the result, in most cases, of insufficient endogenous antithrombotic control of the originally localized hemostatic event. When excess thrombin enters the blood stream or circulating blood is exposed to increasing quantities of thrombin on the “inner side” of the hemostatic plug or the blood clot, the effect of this enzyme can be detrimental. It continues to activate platelets and convert soluble fibrinogen into insoluble fibrin network. Upon flow conditions, in vivo, this “runaway” coagulation and platelet activation process results in the formation of thrombi, which are pathological intravascular entities. Thrombi have also been called “blood clots” by lay people. Thrombi can continue to grow and can entirely block blood flow and cause occlusion of the blood vessel. Vascular occlusions result in reduced blood supply distal to their location and increased blood volume and pressure proximal to their location. Reduction of blood flow due to vascular occlusions in organs can cause metabolic damage or failure and tissue necrosis downstream. This tissue starvation caused by reduced blood flow can cause organ malfunction, both acutely and chronically. A thrombus is neither a blood clot nor a hemostatic plug. Thrombi are formed over time under dynamic pressurized blood flow conditions, where blood flow continuously supplies building elements to the progressive thrombus. Thrombi are thus composed of elements of blood but their elemental composition is vastly disproportional to that of blood. Thrombi are tissue-like objects in the blood vessel that contain up to 100-fold more platelets, and several-fold more fibrin(ogen) than the same volume of blood. Further increases the complexity of thrombosis that the dynamics of formation and composition thus response to treatment of thrombi are different in various arteries, veins, the heart, and small vessels.

### The Pathophysiology of Thrombosis and Hemostasis

### Hemostasis

Hemostasis is a vital function that stops bleeding and protects the integrity of blood circulation on both molecular and macroscopic levels. Injury-related hemostasis is initiated locally when the blood tissue barrier is breached and blood exiting the blood vessels encounters the “foreign” surface of subendothelial and other extravascular matter. Similar processes can occur when “foreign” surface or material, e.g., bacteria or cellular debris, accidentally enter the blood stream from the outside. Molecular recognition of the new environment results in chemical and cellular reactions that are geared towards sealing the breach and reach hemostasis. Platelet and thrombin (factor IIa, FIIa) activity is critical to hemostasis. Thrombin is the product of the enzyme cascade that is activated following injury. Thrombin activates platelets and generates fibrin that are essential building elements of the hemostatic plug. The hemostatic plug is responsible for sealing the vascular breach. Complete absence of thrombin or platelets causes paralysis of hemostasis and leads to lethal hemorrhage.

### Hemorrhage

Failure of hemostasis due to inherited (hemophilias, platelet disorders) or acquired (drugs, vitamin deficiency, etc.) disorders causes bleeding. Anticoagulants or antiplatelet agents disable hemostasis and therefore bleeding is their most common adverse effect. This poor hemostatic safety is typical of all antithrombotic drugs in clinical use today. The detrimental antihemostatic and beneficial antithrombotic effects of both direct and indirect anticoagulants require extremely careful balancing of the dose by the physician. All anticoagulants in medical practice can cause life-threatening bleeding at their most efficacious doses. For the purposes of this application, the most efficacious dose is defined as a limiting dose level that is just below the dose levels that do not produce additional increase in antithrombotic efficacy. Apart from a limited number of specific medical situations, anticoagulants have not been tested or used at their most efficacious doses in the clinic.

### Thrombosis

Thrombosis, just like hemostasis, is a platelet and thrombin dependent process. Thrombosis is a pathological, intravascular, thrombin-dependent, progressive deposition of polymerized fibrin and activated platelets that causes occlusion of blood vessels in various organs. In healthy mammals, intravascular coagulation is localized to the site of hemostasis. Intraluminal progression into thrombosis is efficiently blocked by natural antithrombotic enzymes and inhibitors, such as activated protein C, plasmin, and antithrombin. Thrombosis develops when the antithrombotic system fails to control further intravascular thrombin generation. Causality of macrovascular and/or microvascular thrombosis in morbidity and mortality has been directly documented in various diseases that include deep vein thrombosis, pulmonary thrombo-embolism, peripheral artery thrombosis and embolism, retina vein thrombosis, as well as myocardial infarction (Meadows, Med J Aust. 1965; 4:409-11; Harland, Lancet. 1966; 26:1158-60), ischemic stroke (Carmon, J Neurol Sci. 1966; 4:111-9), anthrax sepsis (Dalldorf, J Neurol Sci. 1966; 4:111-9), meningococcal sepsis (Dalidorf, Arch Pathol Lab Med. 1977; 101:6-9), or heparin-induced thrombocytopenia (Rhodes, Surg Gynecol Obstet. 1973; 136:409-16), each the entirety of which is herein incorporated by reference. Evidence for organ damage of thrombotic occlusion and hypoxia origin is also available in other disease groups, such as hemorrhagic fevers (Dennis, Br J. Haematol. 1969; 17:455-62; Gear, Rev Infect Dis. 1979; 1:571-91; Ignatiev, Immunol Lett. 2000; 71:131-40), diabetic angiopathy (Ishibashi, Diabetes. 1981; 30:601-6; Boeri, Diabetes. 2001; 50:1432-9), kidney disease (Miller, Kidney Int. 1980; 18:472-9; McCutcheon, Lupus. 1993; 2:99-103), each the entirety of which is herein incorporated by reference, and several other conditions.

### Current Treatment of Thrombosis

Although thrombosis and hemostasis are not identical molecular processes, they are similar enough that antithrombotic drugs developed to date inadvertently target both. Thrombosis is treated with antiplatelet, profibrinolytic, and anticoagulant agents, yet most of these agents can completely block both thrombosis and hemostasis when administered at their maximally effective doses. Antithrombotic drugs either target the building blocks of thrombi (fibrin and platelets) or inhibit molecules (coagulation factors) and cells (platelets) from participating in the thrombus-forming process. It is widely believed among clinicians and researchers that if an antithrombotic agent is unable to block hemostasis it will not work in thrombosis.

One of the oldest anticoagulant antithrombotic agents, heparin, is still the most widely given injection in the world. Sufficiently high doses of heparin can achieve nearly 100% efficacy but only at the cost of paralyzing hemostasis at such doses. Unfortunately, newer antithrombotic agents, such as fractionated heparins or direct thrombin inhibitors agents do not fare much better. As a result, antithrombotic agents, especially anticoagulants and profibrinolytic agents, must be administered at less than their maximally efficacious doses, and thrombosis remains an under-treated disease. Introduction of new compounds that are based on the promise of improved efficacy but are unable to promise improvement of hemostatic safety is unjustifiable. To date, antithrombotic compounds fall short of promising improvement of safety. The ideal antithrombotic agent will anti-coagulate circulating blood without adversely affecting hemostasis.

There remains a pressing medical need for the development of safe yet efficacious agents that block intravascular thrombin generation without paralyzing hemostasis. Antithrombotic agents in use or under development, such as sulfated glycosaminoglycans (e.g., heparins), vitamin K antagonists (e.g., coumarins), inhibitors of coagulation factors I, II, V, VIII, TFFVII, IX, and X, antiplatelet agents (e.g., clopidogrel), profibrinolytic agents (e.g., streptokinase) and the like, when given at their most efficacious doses, disable hemostasis and cause bleeding. Thus individuals receiving these and other antithrombotic agents at submaximal doses are still at increased risk of hemorrhage, yet remain inadequately protected from thrombosis.

### OBJECTS AND SUMMARY OF THE INVENTION

It is an object of this application to overcome the objections deficiencies and drawbacks of the prior art. It is also an object of this invention to provide a class of compounds that both selectively reduce intravascular thrombin-generating thromboplastin antecedent activity and do not paralyze hemostasis at any dose level.

It is another object of this invention to provide superior hemostatic safety and preferred prophylactic or therapeutic use of antithrombotic compounds versus equiefficacious doses of antithrombotic agents that can cause harm by disabling hemostasis at efficacious or supraefficacious doses.

It is another object of this invention to provide antithrombotic compounds for medicinal use of in a subject including a human or veterinary patients, who is at risk or suffers from a vaso-occlusive disease, characterized by intravascular thrombin activity-related obstruction of arteries, veins, or microvessels, and would benefit from the use of a hemostatically safe anticoagulant agent.

It is another object of this invention to provide antithrombotic compounds having improved hemostatic safety and antithrombotic effectiveness of compounds when used in combinations with other preventive and treatment modalities that target conditions characterized by vaso-occlusive diseases or conditions that are associated with increased risk of vaso-occlusive diseases.

It is another object of this invention to provide antithrombotic compounds that have biologically significant inhibitory activity against intravascular thrombin-generating thromboplastin antecedent activity but not against other proteins or enzymes of the extrinsic and/or common coagulation pathways, including coagulation factors I, II, V, VII, VIII, IX, X, and tissue factor, in humans.

It is another object of this invention to provide hemostatic safety of antithrombotic compounds targeting FXI that when used in pharmaceutically acceptable formulations at doses that inhibit at least 20% and as much as 100% of the intravascular thrombin-generating thromboplastin antecedent activity but less than the corresponding degree of inhibition of the hemostatic activity of other components the hemostatic system when administered to a human.

It is another object of this invention to provide antithrombotic compounds in pharmaceutically acceptable formulations with at least 10% better hemostatic safety than that of equiefficacious doses of other direct and indirect inhibitors of vascular occlusions.

It is a feature of the invention to provide antithrombotic compounds that can be selected from any of the following categories: a) small molecule enzyme inhibitors that interfere with or block the enzymatic activity of activated thromboplastin antecedent, when delivered to a human by pharmaceutically acceptable formulations and means. b) neutralizing antibodies that inhibit activated thromboplastin antecedent activity or thromboplastin antecedent activation, when delivered to a human by pharmaceutically acceptable formulations and means. c) polypeptides that interfere with activated thromboplastin antecedent activity or thromboplastin antecedent activation, when delivered to a human by pharmaceutically acceptable formulations and means. d) peptidomimetics and small molecules that interfere with or block activated thromboplastin antecedent activity or thromboplastin antecedent activation, when delivered to a human by pharmaceutically acceptable formulations and means. e) nucleic acid, DNA or RNA analogs that interfere with or block factor activated thromboplastin antecedent, thromboplastin antecedent activation, or thromboplastin antecedent production, or increase elimination of thromboplastin antecedent from the circulation when delivered to a human by pharmaceutically acceptable formulations and means. f) thromboplastin antecedent, thrombin, or FXII analogs, related peptides and peptidomimetics that interfere with or block activated thromboplastin antecedent activity or thromboplastin antecedent activation, in vivo, when delivered to a human by pharmaceutically acceptable formulations and means.

It is another object of this invention to provide an antithrombotic compound that is a dodecapeptide having the amino acid sequence of Glu-Glu-Val-Ala-Asn-Ala-Trp-Ser-Met-Ser-Pro-Ala, or Glu-Lys-Met-Glu-His-Gly-Ile-Trp-Asn-Arg-Thr-Ala.

It is another object of this invention to provide antithrombotic compounds in pharmaceutical formulations of immediate or extended release, with specific anti thromboplastin antecedent activity, administered in a clinically efficacious dose to a human by enteral and/or parenteral routes to treat or prevent vascular occlusions via reducing thromboplastin antecedent-dependent thrombin generation, in vivo, when delivered to a human by pharmaceutically acceptable means.

It is another object of this invention to provide antithrombotic compounds, the therapeutic usefulness is due to hemostatic safety of compounds in a human or veterinary patient that does not have a condition where an inhibitor of thromboplastin antecedent activity would paralyze coagulation-dependent hemostasis, or where inhibition of thromboplastin antecedent activity would have no circulating substrate, such as thromboplastin antecedent, in vivo.

It is another object of this invention to provide an antithrombotic agent, including: a compound, the compound having an optimal efficacious dose the compound causing a selective reduction of intravascular thrombin-generating thromboplastin antecedent activity at the optimal efficacious dose, and the compound lacking a paralyzing effect on hemostasis at any dose level.

It is another object of this invention to provide a method utilizing an antithrombotic agent of for improving hemostatic safety and prophylactic or therapeutic efficacy in thrombosis treatment and prevention, including the step of substituting at least one of the compounds of claim 1 for equiefficacious doses of antithrombotic agents that can cause harm by disabling hemostasis at efficacious or supraefficacious doses.

It is another object of this invention to provide a medicinal use of antithrombotic compounds including administration of an efficacious dose of at least one of an the compounds to the patient who is at risk or suffers from a vaso-occlusive disease, the vaso-occlusive disease characterized by intravascular thrombin activity-related obstruction of arteries, veins, or microvessels, and would benefit from the use of a hemostatically safe anticoagulant agent.

It is another object of this invention to provide a method utilizing an antithrombotic agent of for improving hemostatic safety and prophylactic or therapeutic efficacy in thrombosis treatment and prevention, including the step of using the compounds in combinations with other preventive and treatment therapies that target conditions characterized by vaso-occlusive diseases or conditions that are associated with increased risk of vaso-occlusive diseases.

It is another object of this invention to provide antithrombotic compounds that lack a paralyzing effect on hemostasis at any dose level specifically lack any effect against other proteins or enzymes of the extrinsic and/or common coagulation pathways, the other proteins or enzymes namely being coagulation factors I, II, V, VII, VIII, IX, X, and tissue factor.

It is another object of this invention to provide antithrombotic agents with selective reduction of intravascular thrombin-generating thromboplastin antecedent activity at the optimal efficacious dose inhibits at least 20% and as much as 100% of the intravascular thrombin-generating thromboplastin antecedent activity; and, the lacking a paralyzing effect on hemostasis equates to a maximum inhibition of less than 80% inhibition of the hemostatic activity of the homeostatic system.

It is another object of this invention to provide antithrombotic compounds in pharmaceutically acceptable formulations with at least 10% better hemostatic safety than that of equiefficacious doses of other direct and indirect inhibitors of vascular occlusions.

It is another object of this invention to provide hemostatic safety of the effective dosage forms of antithrombotic agents where a property of the agent equates to at least one of: specific inhibition of activated thromboplastin antecedent, in vivo, specific inhibition of thromboplastin antecedent activation, in vivo, specific inhibition of thromboplastin antecedent production, in vivo, enhancement of thromboplastin antecedent elimination, in vivo, and enhancement of activated thromboplastin antecedent elimination, in vivo.

It is another object of this invention to provide antithrombotic agents wherein the agent is at least one compound selected from at least one of the categories consisting of: a) small molecule enzyme inhibitors that interfere with or block the enzymatic activity of activated thromboplastin antecedent, when delivered to a human by pharmaceutically acceptable formulations and means; b) neutralizing antibodies that inhibit activated thromboplastin antecedent activity or thromboplastin antecedent activation, when delivered to a human by pharmaceutically acceptable formulations and means; c) polypeptides that interfere with activated thromboplastin antecedent activity or thromboplastin antecedent activation, when delivered to a human by pharmaceutically acceptable formulations and means; d) peptidomimetics and small molecules that interfere with or block activated thromboplastin antecedent activity or thromboplastin antecedent activation, when delivered to a human by pharmaceutically acceptable formulations and means; e) nucleic acid, DNA or RNA analogs that interfere with or block factor activated thromboplastin antecedent, thromboplastin antecedent activation, or thromboplastin antecedent production, or increase elimination of thromboplastin antecedent from the circulation when delivered to a human by pharmaceutically acceptable formulations and means; and, f) thromboplastin antecedent, thrombin, or FXII analogs, related peptides and peptidomimetics that interfere with or block activated thromboplastin antecedent activity or thromboplastin antecedent activation, in vivo, when delivered to a human by pharmaceutically acceptable formulations and means.

It is another object of this invention to provide a antithrombotic compound that is a dodecapeptide having the amino acid sequence of Glu-Glu-Val-Ala-Asn-Ala-Trp-Ser-Met-Ser-Pro-Ala, or Glu-Lys-Met-Glu-His-Gly-Ile-Trp-Asn-Arg-Thr-Ala.

It is another object of this invention to provide antithrombotic agents wherein at least one agent is at least one of an immediate release pharmaceutical formulation, an extended release pharmaceutical formulation, administrable in a clinically efficacious dose to a patient by the enteral route and administrable in a clinically efficacious dose to a patient by the parenteral route.

It is another object of this invention to provide a pharmaceutically acceptable preparation of an antithrombotic non-antihemostatic compound, comprising at least one active molecule, the at least one active molecule having at least the properties of: rendering blood coagulation factor XI unable to participate in a reaction that is part of any thrombus producing pathway, and at least one of exerting no effect on any other blood coagulation factor's participation in the any thrombus producing pathway and exerting an enhancing effect on at least one other blood coagulation factor's participation in the any thrombus producing pathway.

It is another object of this invention to provide class of active antithrombotic preparations wherein at least one active molecule in the preparation is selected from the group consisting of: a) a small molecule non-peptide enzyme inhibitor that specifically at least one of interferes with the enzymatic activity of FXIa, blocks the enzymatic activity of FXIa, interferes with the activation of FXI, blocks the activation of FXI, interferes with the production of FXI, and blocks the production of FXI; b) an antibody or having a binding specificity for at least one of FXI, FXIa, and an FXI activating enzyme; c) an antibody fragment having a binding specificity for at least one of FXI, FXIa, and an FXI activating enzyme; d) an enzyme-antibody chimera having a binding specificity for at least one of FXI, FXIa, and an FXI activating enzyme; e) an enzyme-antibody fragment chimera having a binding specificity for at least one of FXI, FXIa, and an FXI activating enzyme; f) a polypeptides that at least one of interferes with FXIa activity and interferes with FXI activation; g) a peptidomimetic that specifically at least one of interferes with the enzymatic activity of FXIa, blocks the enzymatic activity of FXIa, interferes with the activation of FXI, blocks the activation of FXI, interferes with the production of FXI, and blocks the production of FXI; h) a small molecule peptide that specifically at least one of interferes with the enzymatic activity of FXIa, blocks the enzymatic activity of FXIa, interferes with the activation of FXI, blocks the activation of FXI, interferes with the production of FXI, and blocks the production of FXI; i) one of a Nucleic acid, a DNA analog and an RNA analog, that at least one of directly interferes with the production of FXI, directly blocks the production of FXI, encodes a gene product that interferes with the production of FXI, encodes a gene product that blocks the production of FXI, encodes a gene product that interferes with the enzymatic activity of FXIa, encodes a gene product that blocks the enzymatic activity of FXIa, encodes a gene product that interferes with the activation of FXI, and encodes a gene product that blocks the activation of FXI; j) a sequestration molecule that at least one of specifically isolates FXI, specifically aggregates FXI, specifically isolates FXIa, and specifically aggregates FXIa, k) an inactive analog of one of FXI, thrombin, and FXII that specifically at least one of interferes with the enzymatic activity of FXIa, blocks the enzymatic activity of FXIa, interferes with the activation of FXI, blocks the activation of FXI, interferes with the production of FXI, and blocks the production of FXI; and l) an inactive fragment of one of FXI, thrombin, and FXII that specifically at least one of interferes with the enzymatic activity of FXIa, blocks the enzymatic activity of FXIa, interferes with the activation of FXI, blocks the activation of FXI, interferes with the production of FXI, and blocks the production of FXI.

It is another object of this invention to provide antithrombotics that include devices having a surface adaptable to being exposed to circulating blood, and at least one active antithrombotic molecule targeting FXI attached to the surface.

It is another object of this invention to provide a method utilizing the antithrombotic of this application for preventing a thromboses or thrombin-dependent vaso-occlusive diseases resulting from a medical/surgical procedure, including the steps of: administering at least one sufficiently efficacious dose of the at least one active molecule of claim 1 sufficiently prior to, the initiation of the medical/surgical procedure to allow the at least one active molecule to significantly reduce a circulating concentrations of activatable FXI; and, continuing to administer doses, of the at least one active molecule, sufficiently efficacious to maintain the significantly reduced circulating concentrations of activatable FXI, until sufficient time has elapsed that the thrombosis or thrombin-dependent vaso-occlusive diseases are no longer a threat.

It is another object of this invention to provide a diagnostic assay utilizing the antithrombotic compounds of this application, including: an agent to block the intrinsic pathway; one of the antithrombotic compounds that directly blocks the activation of FXI or the activity of FXIa; a standard of at least one known concentration of activatable FXI, the standard having a similar composition to that of samples to be tested; and, a diluent for diluting the samples, the agent to block, the antithrombotic compounds, and the standard.

It is another object of this invention to provide a method for diagnosing predisposition for developing a thromboses or thrombin-dependent vaso-occlusive disease, including the steps of: determining a circulating FXI concentration for a patient; applying a correlation algorithm to the circulating FXI concentration, the correlation algorithm being derived from a statistically determined population database of paired datum of a first datum for a survey subject's the circulating FXI concentration and a second datum for the survey subject's incidence of developing a thromboses or thrombin-dependent vaso-occlusive disease; and, reading out the result from the algorithm of the patient's incidence of developing a thromboses or thrombin-dependent vaso-occlusive disease that correlates with the patient's the circulating FXI concentration.

In summary, the present invention relates to methods, compounds, their pharmaceutically acceptable analogs, isomers, salts, hydrates, solvates and prodrug derivatives, and pharmaceutically acceptable compositions thereof that have particular biological properties. These compounds are useful because they selectively reduce intravascular factor XI activity, which results in a safe antithrombotic effect. In another aspect, the invention relates to methods of using these inhibitors as therapeutic agents in humans which have, or are at risk of, developing vaso-occlusive diseases, such as myocardial infarction, stroke, restenosis after angioplasty, thrombotic diseases, and alike. The present invention also includes pharmaceutical compositions comprising a hemostatically safe and pharmaceutically effective amount of the compounds of this invention and a pharmaceutically acceptable carrier. In yet another aspect, the present invention includes methods comprising using compounds and pharmaceutical compositions of this invention for preventing or treating disease states characterized by thrombus formation or pathological intravascular blood coagulation in humans. Optionally, the methods of this invention comprise administering the pharmaceutical composition in combination with an additional therapeutic agent such as another antithrombotic, antiplatelet, thrombolytic, or anticoagulant agent. The preferred compounds also include their pharmaceutically acceptable analogs, isomers, hydrates, solvates, salts and prodrug derivatives. Finally the present invention includes the use of the above compounds in diagnostic assays for assessing the risk of developing a thrombotic disease condition or evaluating the effectiveness of treatment. Lastly, it is also contemplated that FXI assays utilizing the above compounds as well as other FXI assay techniques are useful as a screening assay for finding additional members of this class of therapeutics.

The above, and other objects, features and advantages of the present invention will become apparent from the following description read in conjunction with the accompanying drawings.

### BRIEF DESCRIPTION OF THE DRAWINGS

FIG. 1. A block diagram illustrating the structural and compositional elements of the normal state of the circulatory system (Homeostasis) with regards to its blood coagulation potential.

FIG. 2. A block diagram illustrating the structural and compositional elements of Hemostasis

FIG. 3. A block diagram illustrating the structural and compositional elements of Thrombosis

FIG. 4. A block diagram illustrating the effect of inhibition or absence of FII on hemostasis and thrombosis

FIG. 5. A block diagram illustrating the effect of inhibition of absence of FV or FX on hemostasis and thrombosis

FIG. 6. A block diagram illustrating the effect of inhibition or absence of FVII on hemostasis and thrombosis

FIG. 7. A block diagram illustrating the effect of inhibition or absence of FVIII on hemostasis and thrombosis

FIG. 8. A block diagram illustrating the effect of inhibition or absence of FXI on thrombosis and hemostasis

FIG. 9. A block diagram illustrating the effect of inhibition or absence of FXII on thrombosis and hemostasis

FIG. 10. A block diagram illustrating the effect of inhibition or absence of FIX on hemostasis and thrombosis

FIG. 11. A block diagram illustrating the effect of inhibition or absence of platelets on thrombosis and hemostasis

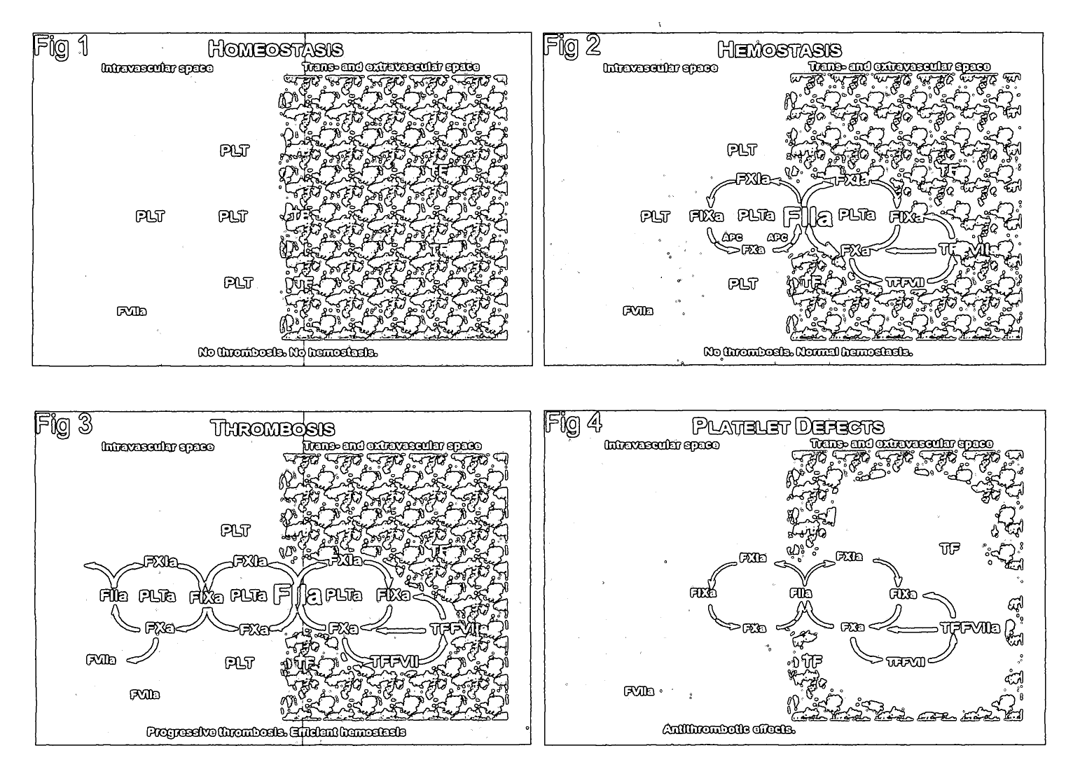

### DEFINITIONS

For purposes of interpreting the meaning and scope of the claims and specification of this application the following definitions govern:
antihemostatic effect—adverse attribute of a pharmaceutical agent or therapeutic modality that is characterized by interference with normal hemostasis during effective treatment of a human
antithrombotic efficacy—beneficial attribute of a pharmaceutical agent or therapeutic modality that is characterized by prevention, halting the progression of, or reversal of thrombosis in a human
blood clot—viscous gel formed of and containing all components of blood in proportion to liquid blood
coagulation—transformation of blood from liquid to gel phase as a result of polymerization of fibrin monomers
common coagulation pathway—interactions of blood coagulation proteins V, VIII, IX and X that lead to the generation of thrombin (factor IIa)
efficacious dose—the dose of a pharmaceutical agent that results in a clinically demonstrable effect in the prevention or treatment of a human disease
equiefficacious dose—a specific dose of a pharmaceutical agent that results in the same prophylactic or therapeutic effect as a specific dose of another pharmaceutical agent in humans
extrinsic pathway—interactions of blood coagulation proteins that lead to the generation of coagulation factor Xa and/or IXa without involvement of thromboplastin antecedent (factor XI)
hemorrhage—clinically manifest internal or external bleeding following transvascular injury and failure of hemostasis
hemostasis—coordinated defense mechanism aimed at preserving the integrity of blood circulation
hemostatic safety—the attribute of a pharmaceutical agent or modality that is characterized by preservation of hemostasis during effective treatment of a human
homeostasis—normal state of circulating blood characterized by insignificant enzymatic activity, such as thrombin generation and platelet activation
inhibitor—a pharmaceutical agent or modality that inhibits the physiological or pathological function or activity of a protein or proteins
intrinsic pathway—interactions of blood coagulation proteins that lead to the generation of coagulation factor IXa without involvement of coagulation factor VIIa.
thromboplastin antecedent—a circulating blood coagulation zymogen, also termed coagulation factor XI, which, in its activated enzyme form, factor XIa, can proteolytically activate factor IX
supraefficacious dose—dose of a therapeutic agent that is higher than needed to achieve a clinically defined level of prophylactic or therapeutic efficacy
thrombin—also termed coagulation factor IIa, the key multifunction enzyme of hemostasis that is responsible, among others, for converting fibrinogen to fibrin and activating platelets
thrombin-dependent vaso-occlusive diseases—pathological processes in the circulation where thrombin generation plays a pivotal role in the development of vascular occlusions, e.g. coronary thrombosis, restenosis following angioplasty, deep vein thrombosis, disseminated intravascular coagulation in sepsis, etc.
thrombosis—diseases caused by the blockage of the blood flow to and from organs by thrombi
thrombus/thrombi—pathological intravascular tissue-like fibrin/platelet mass that also contains other blood components
usefulness—the characteristic of a pharmaceutical agent or modality who's use results in a net benefit to humans

### DETAILED DESCRIPTION OF THE PREFERRED EMBODIMENT(S)

### Rationale of the Present Invention

The present invention is a class of antithrombotic agents that specifically target blood coagulation factor XI. The clinical phenotype of various inherited coagulation disorders that cause bleeding and the clinical experience obtained during treatment and overdose of patients with anticoagulants, such as warfarin, that result in severe hemorrhage help to understand the rationale for this invention. These clinical observations help to understand why reducing FXI activity offers a solution to the problem of drug-induced bleeding as outlined above.

The extrinsic and intrinsic blood coagulation pathways converge at the level of blood coagulation factor IX (FIX) (FIGS. 2 and 3). FIX is cleaved and thus activated to FIXa by both TFFVIIa and factor XIa. FIXa, in complex with FVIIa, then activates FX to FXa. TFFVIIa also activates factor X (FX) to FXa. FXa in complex with FVa then converts prothrombin into thrombin, the key enzyme of both thrombosis and hemostasis (FIG. 3).

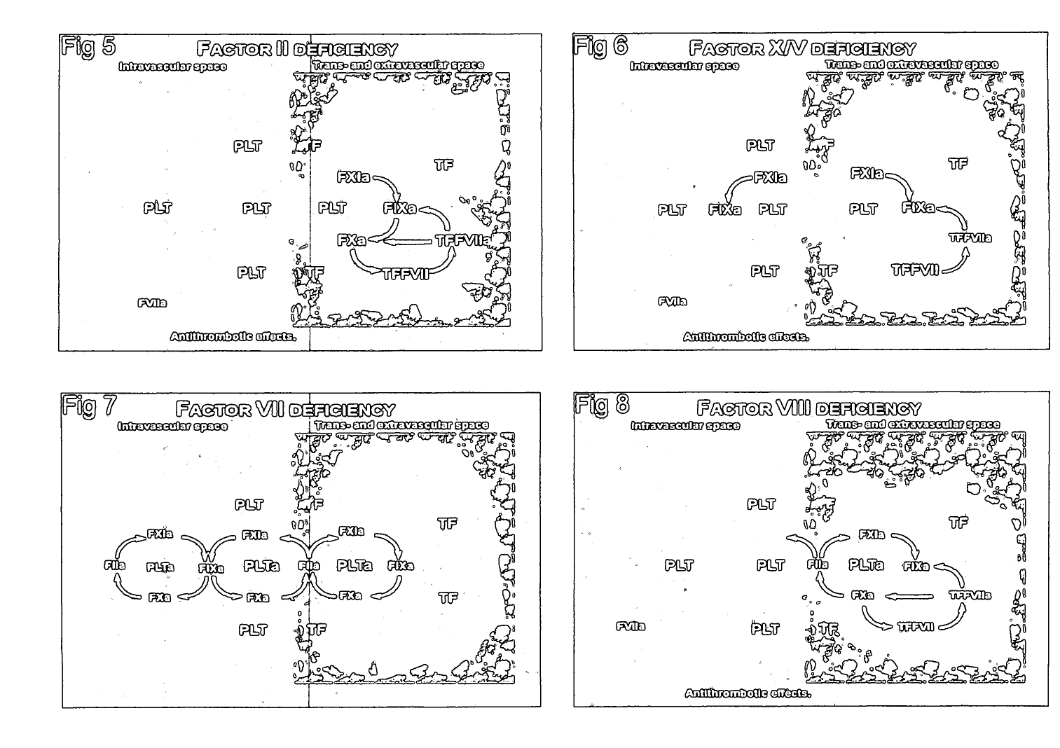

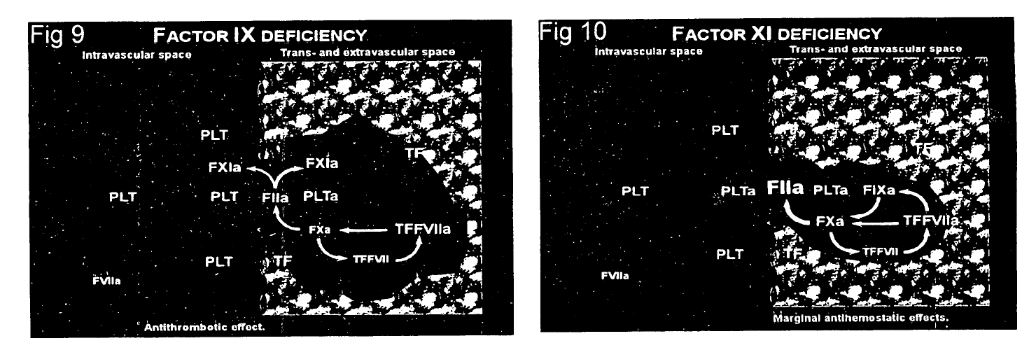

Absence of FII activity (FIG. 4), factor X activity (FIG. 5), factor VII activity (FIG. 6), and factor IX activity (FIG. 7), whether due to absence of the coagulation factor, absence of the cofactor, or due to the presence of a specific inhibitor of these coagulation factors cause symptomatic hemophilia, or even lethal bleeding. Absence of platelets or platelet function also causes severe bleeding (FIG. 8). In sharp contrast, absence of FXI activity causes either mild hemophilia or, in majority of the cases, no bleeding symptoms (FIG. 9). It is thus clear that activation of FIX by TFFVIIa is the first key enzyme reaction and cleavage of fibrinogen by thrombin is the last key enzyme reaction in maintenance of hemostasis. Any agent that blocks the enzymes and their cofactors between these levels is ultimately capable of disabling hemostasis. At present, all anticoagulant drugs in use target the coagulation cascade at or under the level of TFFVII and/or FIX. It is thus not surprising that all can produce severe bleeding, just like symptomatic hemophilias.

Based on the above described and illustrated assessment of the clinical symptoms of inherited or acquired coagulation factor deficiencies, the only remaining rational choice for the safe therapeutic alternative to all existing modalities is specific inhibition of FXI activity. This modality has low hemostatic risks because it cannot disable hemostasis (FIG. 8). Thromboplastin antecedent (factor XI, FXI) is a circulating zymogen that can become proteolytically activated by thrombin, FXIa, and FXIIa. FXI is also present on platelets and platelets are important to localizing FXI activity. For example, efficient activation of FXI to FXIa by thrombin requires the presence of platelets, and is key to the intravascular progression of thrombosis. FXIa contributes to thrombin generation and thus thrombosis by activating coagulation factor IX in the presence of platelets. Activation of FXI, however, is not essential to hemostasis.

Inhibition or lack of various coagulation proteins or platelets have the clinical appearance of the corresponding forms of hemophilias and platelet disorders. Complete absence of most of the coagulation factors or platelets is incompatible with normal life. In contrast, untreated complete inherited FXI deficiency is compatible with normal life in most cases. Most affected subjects with the deficiency remain asymptomatic, and some present with symptomatic hemophilia C, the mildest form of hemophilias. Furthermore, there is a marked lack of correlation between the level of circulating FXI in deficient individuals and the expression of hemophilia C symptoms. Consequently it has been proposed that symptomatic hemophilia C requires another mild coagulation deficiency in addition to the FXI deficiency and it is a common occurrence that an adult with inherited hemophilia C is not diagnosed until a hemorrhage event occurs during surgery performed on a highly vascularized tissue. Hemophilia C, when symptomatic, is a mild bleeding disorder that has been extensively described in humans and does not reduce life expectancy. It is endemic in certain breed of cattle (Holstein) and dogs, causing no apparent problems. FXI deficiency has also been studied in FXI−/− knock-out mice that appear to be asymptomatic for life, Gailani, Blood Coagul Fibrinolysis. 1997 March; 8(2):134-44, the entirety of which is incorporated herein by reference.

It is clear from clinical and experimental observations that even severe FXI deficiency alone does not entirely disable hemostasis. During monospecific inhibition of FXI by compounds of this invention there is no significant coagulation within the blood vessel under flow conditions but any blood that comes in contact with surface-bound TF while exiting the blood vessel and passing through extravascular tissues will readily clot. The reason is that TFFVIIa can sufficiently accelerate the generation of both thrombin (FIIa) and TFFVIIa via both FIX and FX activation to compensate for the absence of FXI feedback activation and acceleration of FIX activation (FIG. 8). As a result, treatment with an inhibitor of FXI will result in an antithrombotic effect with significantly improved hemostatic safety.

The present discovery that thrombosis is a factor XI-dependent event and thus can be safely prevented or treated by targeting FXI provides another aspect to the underlying rationale behind the present invention. Progression of primary hemostatic plugs or small clots into thrombi likely depends on the intrinsic coagulation cascade via thrombin catalyzed activation of FXI in the presence of activated platelets (FIG. 8). Thrombin leaking from the initial clot is the key culprit in thrombus propagation. Formerly thrombogenic clots can be “neutralized” and converted into non-thrombogenic clots by local treatment of the clot with hirudin or PPACK, both virtually irreversible inhibitors of thrombin. Since neither inhibitors affect TF, TF is already sealed away and appears to be irrelevant in the progression phase of thrombosis. Circulating blood that supplies the building blocks (platelets) and mortar (fibrin) to the structure of thrombi does not contain appreciable quantities of TF to promote intravascular coagulation. Moreover, thrombogenic surfaces, whether bacteria, subendothelial matter, intravascular devices, or prosthetic vascular grafts, quickly become covered by virtually impenetrable layers of fibrin/platelet conglomerates that do not serve as continued sources of TF or thrombogenic “foreign” surface under flow conditions. Accordingly, the pathological process of intravascular thrombin generation is the result of poorly controlled runaway feed back activation of FXI. When FXI activity is reduced, the intravascular component of the coagulation cascade is efficiently inhibited. Accordingly, thrombosis is also reduced, since its progression primarily depends on activation of FXI (FIG. 8). This modality prevents the potential of or stops runaway intravascular thrombin generation while leaving the local extra-endothelial surface-dependent defense mechanism of the extrinsic TFFVII pathway unaffected. Accordingly, this invention teaches that inhibition of FXI activity is a safe intravascular antithrombotic antithrombin modality.

### Usefulness of Methods and Compounds of the Invention

Claims of usefulness of new anticoagulants are typically based on anticipation of clinical efficacy. However, even one skilled in the art cannot anticipate, based on in vitro studies, the usefulness of anticoagulants with certainty. Unfortunately, the antithrombotic effects of a compound in typical animal models of thrombosis does not predict its usefulness in patients either. The reason is that existing animal models are not suitable for determining hemostatic safety of efficacious doses of anticoagulants in human thrombotic diseases. For example, warfarin treatment of animals producing INR in the range of 2 to 3, or heparin treatment producing an APTT prolongation of 1.5 to 2.5-fold baseline does not produce bleeding complications in animal models. Yet, these treatments at comparable INR and APTT values are efficacious and produce hemorrhage in human patients. It should thus be obvious for those skilled in the art that no in vitro study, such as measurement of prolongation of clotting time, and no animal model are acceptable as reliable and predictive of evidence, or even indication, of the superior safety of reducing FXI activity versus equiefficacious doses of a comparator anticoagulant, e.g., heparin. Usefulness, however, can be anticipated based on theoretical considerations and rationale, such as in the case of this invention. Case by case evidence of usefulness, that is safety of the efficacious dose, is for obtained in prospectively designed controlled clinical trials that are powered to demonstrate improvement of safety versus another selected antithrombotic agent in patients with selected disease conditions.

This invention proposes a solution to the problem of improving the safety of anticoagulant therapy through monospecific and exclusive reduction of circulating factor XI activity. Monospecificity is important to the usefulness of this modality. Enzyme inhibitors that had been grouped together and deemed useful because they can inhibit the activity of several coagulation enzymes sharply contrast the spirit of this invention and obviously defeat the intention of the inventor of this invention of improving hemostatic safety of anticoagulation in human subjects. No prior art targets an inhibitor specific for FXI as superior and more useful compared to inhibition of other enzymes. More specifically, inhibitors of groups of compounds that are deemed useful for their ability to inhibit the functions of platelets, factors I, II, V, VII, VIII, IX, X, XIII, kallikrein, or TF are not useful as hemostatically safe antithrombotic agents. Such compounds would not be as safe as specific inhibitors of FXI, and thus would be considered less useful. In fact, this invention demonstrates that the most efficacious dose of an inhibitor specifically targeted for any other single factor, other than FXI, would certainly result in clinical symptoms of severe hemophilia.

Not all anticoagulant agents are antithrombotic, and not all antithrombotic agents are anticoagulant. Anticoagulants might prove to be useful if they do not have unexpected side effects, if they can be delivered and administered in efficacious dosage forms, if the antithrombotic benefits outweigh the risk of antihemostatic effects, and if the formulation or compound have the necessary duration of activity as administered. It is well known to those skilled in the art that anticoagulants are not necessarily useful as safe antithrombotic agents when used at doses that cause measurable anticoagulation and inhibit thrombosis, in vivo. Examples of unsafe anticoagulants that are currently used only either ex vivo or in vitro due to non-coagulation-specific and coagulation-specific potentially lethal toxicity include, among others, citrate, EDTA, oxalate, PPACK, and benzamidine. Examples of anticoagulants with coagulation-specific potential for lethal toxicity at their most efficacious doses, (some still currently in clinical use or under testing) include, among others, heparins, direct antithrombins, coumarins, hirudins, FIX inhibitors, FX inhibitors, and activated protein C. These agents carry the risk of actually killing the patient when used at their most efficacious antithrombotic dosage range. The class of compounds of the present invention are fundamentally different from all the above because they cannot produce coagulation-specific lethal toxicity due to paralysis of hemostasis at their most efficacious doses.

Targeting FXI as an antithrombotic approach has historically been counter-intuitive. At the time conceptualizing and filing this invention, there was no data available indicating that inhibition of FXI would be antithrombotic. Since there hardly are any bleeders among hemophilia C patients, most assumed that blocking FXI activity alone would have no significant antithrombotic effect since there appears to be no significant antihemostatic effect. Because of the general prejudice against the therapeutic potential of a FXI inhibitor, the focus of pharmaceutical development and discovery of novel antithrombotic agents has consequently targeted other coagulation proteins, or several coagulation proteins at the same time.

### Compounds of the Present Invention

Generally, the specific FXI inhibitor compounds or modalities that are considered useful according to the present invention are differentiated from other compounds by better hemostatic safety when administered at equiefficacious doses. Such compound can be any agent of the following groups:

a) Small molecule non-peptide enzyme inhibitors that interfere with or block the enzymatic activity of FXIa or the activation of FXI. These compounds are predominantly direct competitive or non-competitive enzyme inhibitors.
b) Antibodies, including neutralizing antibodies or antibody fragments that inhibit FXIa activity or FXI activation, or enhance clearance of the antibody-bound FXI molecule from the circulation, labeling antibodies that mark FXI as junk protein for uptake and/or degradation, and chimeric antibodies which utilize the antibody's specificity but add other functionality to the compound, such as proteolytic or co-enzymatic activity. Antibodies that are useful as therapeutic agents in humans, under the terms of this invention, are preferably recombinant humanized monoclonal antibodies or antibody fragments.
c) Polypeptides that interfere with FXIa activity or FXI activation. These peptides are preferably analogs of parts of naturally occurring human proteins without significant immunogenicity (see examples).
d) Peptidomimetics and small molecules that interfere with or block FXIa activity or FXI activation. These molecules can act against the active center, other epitopes and domains of FXI, FXII and thrombin but only block the function of FXI, in vivo.
e) Nucleic acid, DNA or RNA analogs that interfere with or block FXIa activity, FXI activation, or FXI production. These molecules can both directly inhibit FXI activity or activation, and block the expression of FXI, such as by antisense binding to the FXI gene.
f) Sequestration molecules that specifically isolate or aggregate FXI rendering it unavailable to participate in the coagulation cascade. For example: multivalent FXI binding molecules—natural or synthetic agglutinins that bind FXI without affecting other blood components.
g) FXI, thrombin, or FXII analogs, related peptides and peptidomimetics that interfere with or block FXIa activity or FXI activation—without significant inhibition of other blood coagulation proteins at or below its maximum efficacious dose, in vivo. Included here would be such molecules that mimic binding of FXI to hepatocyte feedback receptors involved in limiting the production of FXI to the optimal concentration in the circulation.
h) Molecules that reduce the biological half life of FXI result in a reduction in the level of the circulating protein. Such compounds enhance the clearance of FXI without producing the active species, FXIa.

This invention also encompasses analogs and prodrug derivatives of the compounds contained herein. The term “analogs” refers to a pharmacologically active molecular structures that bear close similarity to the secondary and tertiary molecular structures of the parent prodrugs or drugs, regardless the degree of similarity in the primary molecular structure (i.e., atoms). Analogs have either similar or identical molecular mechanisms of action to the parent compound, in vivo. Prodrugs require biotransformation to yield the active drug, in vivo.

This invention of improving the safety of antithrombotic therapy by reducing FXI activity also encompasses non-pharmacological methods that can reduce the level of circulating FXI by at least 20 and up to 100% using physical and chemical means, such as factor FXI-specific binding material, antibody-coated surfaces, gels, ex vivo or in vivo FXI traps, and alike that can remove FXI from the blood passing through an extracorporeal device or getting in contact with the FXI-trap placed within the body.

### Pharmaceutical Compositions and Formulations

The most appropriate compositions, formulations, and dosage forms are ultimately used for achieving the desired safety and pharmacodynamic effect in a human. The specific goal is to deliver the active ingredients, derived from the compounds of this invention, to the appropriate molecular and cellular targets resulting a decrease in thrombin-generating factor XI activity. Such targets can be, among others, factor XI, cells that produce or degrade factor XI, molecules that specifically interact with factor XI, or molecules that interact with the compounds of this invention to produce the active ingredient. The list below, therefore does not intent to be complete, and serve as a list of examples only. The compounds of this invention may be utilized in all pharmaceutical compositions and in the process of producing those compositions known to those skilled in the art. The compounds within the scope of this invention, whether end-products or intermediates, may be acidic, basic, or converted to salts of various inorganic and organic acids and bases. Various methods are useful for the preparation of the end-products and are known to in the pharmaceutical industry. These include, among others, immediate and extended-release tablets, capsules, elixirs, solutions, suspensions, suppositories, sterile solutions, infusions, injectable formulations, with or without special delivery devices or formulations, and the like.

Formulations of the compounds of this invention are prepared for storage or administration by mixing the compound having a desired degree of purity with physiologically acceptable carriers, excipients, stabilizers etc., and at the time of the peak effect of the compound. Complete inhibition of the FXI activity will result in maximum possible efficacy, however, less than complete inhibition may also result in maximum efficacy. The route of administration, the therapeutic objectives, and the condition of the patient will influence the range of therapeutically effective dosages.

A typical dosage might range from about 0.0001 mg/kg/day to about 100 mg/kg/day, preferably from about 0.001 mg/kg/day to about 10 mg/kg/day, and more preferably from about 0.01 mg/kg/day to about 1 mg/kg/day. The compounds of this invention may be administered to a human subject in various regimens, ranging from a single bolus dose to continuous treatment without time limit. If given in repeated doses, the dose might be repeated several times a day, week, or year. Other dosage regimens may also be useful, such as those typical of controlled release dosage forms, whether enteral or parenteral, that deliver the useful dose over a longer period than immediate release dosage forms.

### Methods of Use

In practicing the methods of this invention, the compounds of this invention may be used alone or in combination with other therapeutic agents. In certain preferred embodiments, the compounds of this invention may be co-administered along with other compounds typically prescribed for human subjects with vaso-occlusive conditions according to generally accepted medical practice, such as anticoagulant agents, thrombolytic agents, and antiplatelet agents. The compounds of this invention may also be used in combination with agents that are being used for the prevention or treatment of underlying conditions that are typically associated with vascular occlusions, and include, among others, anti-inflammatory agents, antibiotics, antiviral drugs, cholesterol lowering agents, drugs used for the treatment of heart failure, and anticancer agents.

Compounds of the present invention are characterized by their ability to inhibit or entirely block symptomatic vascular occlusions, such as those that occur as a result of thrombus formation, while producing less effect on hemostasis than other antithrombotic agents, such as those that have the ability to disable hemostasis at their maximally efficacious doses. Conditions that are characterized by vascular occlusions and justify treatment or prevention using compounds of this invention include those that involve the arterial, capillary, and venous vasculature. Without restricting the use of the compounds of this invention for the disease of any particular blood vessel, the following conditions describe specific clinical examples where the compounds can be utilized.

In the coronary arteries, occlusive thrombus formation often follows the rupture of atherosclerotic plaque. This occlusion is the major cause of acute myocardial infarction and unstable angina. Coronary occlusions can also occur following infections, inflammation, thrombolytic therapy, angioplasty, and graft placements. Similar principles apply to other parts of the arterial vasculature and include, among others, thrombus formation in the carotid arteries, which is the major cause of transient or permanent cerebral ischemia and stroke. Venous thrombosis often follows stasis, infections, inflammatory reactions, and major surgery of the lower extremities or the abdominal area. Deep vein thrombosis results in reduced blood flow from the area distal to the thrombus and may be provided in immediate, controlled, sustained or timed release formulations. Acceptable carriers or diluents for therapeutic use are well known in the pharmaceutical field, and are nontoxic to the recipients at the dosages and concentrations employed. Subjects in need of treatment, typically humans, using the compounds of this invention can be administered dosages that will provide up to maximal efficacy. The dose and method of administration may vary from subject to subject and be dependent upon such factors as the type of patient being treated, its sex, weight, diet, concurrent medication, overall clinical condition, the particular compounds employed, the specific use for which these compounds are employed, and other factors which those skilled in the medical arts will recognize. Methods of administration anticipated include two major categories, enteral and parenteral. These routes include, among others, intravenously, intraarterially, subcutaneously, intramuscularly, colonically, rectally, nasally or intraperitoneally, employing a variety of dosage forms such as suppositories, implanted pellets or small cylinders, aerosols, oral dosage formulations, and injectable or implantable formulations for systemic or topical delivery with immediate or extended-release, and cutaneous or mucous membrane topical formulations with immediate or extended-release, such as ointments, gels, drops, patches. The compounds of this invention can be incorporated into implants and pumps. The compounds of this invention may also be administered in the forms of systemic or topical (local) liposome delivery systems. The compounds of this invention may also be delivered by coupling to targeting moieties that are well known in the art. Liquid formulations generally are placed into containers with sterile access port. Typical adjuvants, lubricating agents, disintegrating agents, etc., such as, e.g., corn starch, gelatin, microcrystalline cellulose, are known to those skilled in the art. Capsule may contain liquid carriers. Other materials of various types may be used as coatings or as modifiers of the physical form of the dosage unit. All of the above is standard and accepted pharmaceutical practice.

### Dosage and Dosage Forms

Useful doses of each compound of this invention are defined by hemostatic safety parameters that are determined in controlled clinical trials. Safe and therapeutically effective dosages may be approximated by either in vitro or in vivo methods, however, determination of the useful dose is preferably done in patients. For each particular compound of the present invention, individual indication-specific determinations are made to determine the optimal dosage required. In a typical dosage form, about 0.5 to 500 mg of a compound or mixture of compounds of this invention, as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated below is obtained. Typically, applications of a compound are commenced at the dosage level that achieves maximum efficacy at a level of hemostatic safety that is at least 10% better, preferably 20% better, more preferably at least 50% better than equiefficacious doses of other agents used for the corresponding indications in comparable clinical settings. Dosage levels might be decreased if the condition of the patient does not require maximum efficacy any longer. The optimal dosage that is tied to patient safety benefit results in elimination of at least 20% and as much as 100% of the thrombin-generating FXI activity in the circulation. Preferably, the dosage results in at least 40 to 100% inhibition of FXI activity in the circulation. More preferably, the dosage results in at least 60 to 100% inhibition of thrombin generating FXI activity in the circulation predisposes to pulmonary embolism. Pulmonary embolism is a major cause of post-surgical mortality. Disseminated intravascular coagulation (DIC) and acute respiratory distress syndrome (ARDS) where compounds of this invention are useful commonly occur within all vascular systems during bacterial sepsis, entry of foreign material into the blood stream following, e.g., trauma and child birth, immune reactions, inflammation, certain viral infections, certain platelet disorders, and cancer. Disseminated intravascular coagulation is a severe complication of many disease conditions and some drug treatments, including, for example, heparin. Thrombotic consumption of coagulation factors and platelets, and systemic coagulation results in the formation of life-threatening thrombi occurring throughout the microvasculature leading to local or widespread hypoxia and organ failure.

In principle, the compounds of the present invention, selected and used as disclosed herein, are useful for safely preventing or treating disease conditions related to and characterized by intravascular thrombin generation. Treatment will thus be useful, e.g., in (a) the treatment or prevention of acute coronary syndromes including myocardial infarction, unstable angina, refractory angina, occlusive coronary thrombus occurring post-thrombolytic therapy or post-coronary angioplasty, (b) the treatment or prevention of any ischemic cerebrovascular syndrome including embolic stroke, thrombotic stroke, or transient ischemic attacks, (c) the treatment or prevention of thrombosis occurring in the venous system including or pulmonary thromboembolism occurring either spontaneously or in the setting of malignancy, surgery or trauma, (d) the treatment or prevention of any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmaphoresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

### EXAMPLES

As described above, the useful doses, dosage forms, and therapeutic regimens of this invention cannot be experimentally determined in vitro or in animals under conditions that do not exactly model the incidence, characteristics, and severity of bleeding complications of efficacious doses of existing anticoagulants in corresponding diseases conditions in humans. The following examples describe conditions where usefulness of doses, dosage forms, and treatment regimens are established in human subjects based on safety advantages of reducing FXI activity. One of the methods to achieve reduction of FXI activity is the use of FXI inhibitors. In the examples listed herein the term “FXI inhibitor” refer to and can be replaced with other modalities that reduce FXI activity.

### Example of Improved Safety of Reducing FXI Activity Versus Vitamin K Antagonists

Chronic warfarin prophylaxis or treatment of patients with atrial fibrillation is efficacious and reduces the incidence of ischemic stroke. Unfortunately, a small percentage of patients treated with warfarin develop severe, disabling, or fatal bleeding complications, such as hemorrhagic stroke, gastrointestinal bleeding, retroperitoneal bleeding, etc. When a FXI inhibitor therapy is tested versus a warfarin, at least as many patients benefit from the anti FXI treatment than from warfarin. However, at least 10% less patients develop severe bleeding complication with anti FXI treatment than with warfarin treatment. In addition, FXI inhibitor therapy, unlike warfarin therapy, does not require laboratory monitoring. This study is conducted in approximately 10,000 study subjects with atrial fibrillation over time, and is characterized by a minimal length of study in a patient of one month. Other study designs exist. In such comparative studies, patient benefits demonstrate the advantages of reducing FXI activity.

### Example of Improved Safety of Using a FXI Inhibitors Versus Heparin Derivatives

Prophylactic or therapeutic heparin (unfractionated or low molecular weights heparins, dermatan sulfate, other glycosaminoglycans) treatment of patients at risk of or suffering from thrombosis is an efficacious modality. Heparins are useful for the treatment and prevention of surgery-associated deep vein thrombosis and for prevention of vascular occlusions during angioplasty. Unfortunately, a small percentage of patients treated with heparins develop severe, disabling or fatal bleeding or thrombotic complications, such as stroke, gastrointestinal bleeding, retroperitoneal bleeding, surgical blood loss, heparin-induced thrombocytopenia, etc. When a FXI inhibitor therapy is tested versus a heparin, the efficacy of the two methods will be comparable. However, at least 10% less patients will develop bleeding complications and no patients will develop heparin-induced thrombocytopenia with anti FXI treatment when compared to treatment with a heparin. In addition, FXI inhibitor therapy, unlike some heparin therapies, does not require laboratory monitoring. This study is ideally conducted in approximately 8000 patients undergoing surgical procedures that significantly increase the incidence of vascular occlusions, and is conducted over a minimum period of a 2 days. Other clinical study designs exist.

Example of Improved Safety of Using a FXI Inhibitors Versus Heparin Derivatives in Conjunction with Thrombolytic Therapy

Prophylactic or therapeutic heparin treatment of patients during thrombolysis is an efficacious modality. Heparins, including various molecular weight compounds from small pentasaccharides to large polymers, are useful for improving the outcome of this intervention. Unfortunately, a small percentage of patients treated with heparinoids in conjunction with a fibrinolytic agent develop severe, disabling or fatal bleeding or thrombotic complications, such as hemorrhagic stroke, gastrointestinal bleeding, retroperitoneal bleeding, blood loss, heparin-induced thrombocytopenia, etc. When a FXI inhibitor therapy is tested versus a heparin, at least as many patients benefit from the anti FXI treatment than from the heparin. However, at least 10% less patients will develop bleeding complications and no patients will develop heparin-induced thrombocytopenia with anti FXI treatment when compared to heparinoid treatment. In addition, unlike most heparin therapies, FXI inhibitor therapy does not require laboratory monitoring. This study is conducted in approximately 40,000 patients suffering from acute heart attack due to coronary disease or acute ischemic stroke due to cerebrovascular disease, and is conducted over a minimum period of 2 hours. Other study designs exist.

### Example of Improving the Therapeutic Window of Antithrombotic Therapy Versus Existing Alternatives

In this controlled clinical trial, the maximally efficacious dose of anti-FXI treatment is tested against one or all alternative therapies, such as heparins, vitamin K antagonists, direct thrombin inhibitors, thrombolytic agents, and antiplatelet therapies. The administered dose of FXI reducing therapy using one or more compounds of this invention blocks up to 100% of all intravascular thrombin-generating FXI activity in the subject. This protocol results in equal or better effectiveness at comparable or better safety level when compared to alternative antithrombotic therapies. This study is conducted in several thousand patients suffering from or at risk of diseases that might require antithrombotic therapy.

### Example for Making a Compound of the Invention

While in vitro or animal testing are not acceptable tools for establishing the safety thus usefulness of drugs, such as anticoagulants, screening compounds for specificity towards factor XI, thus potential utility in the clinic, is an necessary intermediate step in practicing this invention. In such an effort, several FXI-specific peptides have been created using standard phage display technology. Random peptide-expressing phages that exhibited strong binding to immobilized FXI were propagated and then the selected phage clones were tested for activity against FXI. One example of such a phage is given here. Amino acid sequencing of the peptide expressed by this phage yields Gln-Lys-Met-Gln-His-Gly-Ile-Trp-Asn-Arg-Thr-Ala using standard technology. The phage expressing the peptide is then tested in screening coagulation assays, such as activated partial thromboplastin time (APTT) and prothrombin time (PT). This peptide induced significant prolongation of APTT, a standard screening marker of the activity of the intrinsic pathway. This peptide, and other inhibitory compounds with FXI specificity, such as Gln-Gln-Val-Ala-Asn-Trp-Ser-Met-Ser-Pro-Ala, are considered only as examples of intermediate steps in identifying and preparing compounds of this invention that can exclusively reduce FXI activity. Other methods of screening exist.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

What is claimed is:

1. An antithrombotic agent, comprising:
a compound, said compound having an optimal efficacious dose
said compound causing a selective reduction of intravascular thrombin-generating thromboplastin antecedent activity at said optimal efficacious dose, and
said compound lacking a paralyzing effect on hemostasis at any dose level.

2. A method utilizing the antithrombotic agent of claim 1 for improving hemostatic safety and prophylactic or therapeutic efficacy in thrombosis treatment and prevention, comprising the step of:
substituting at least one of the compounds of claim 1 for equiefficacious doses of antithrombotic agents that can cause harm by disabling hemostasis at efficacious or supraefficacious doses.

3. A medicinal use of the compounds of claim 1 in a subject, human or animal, comprising administration of an efficacious dose of at least one of an said compounds to said subject who is at risk or suffers from a vaso-occlusive disease, said vaso-occlusive disease characterized by intravascular thrombin activity-related obstruction of arteries, veins, or microvessels, and would benefit from the use of a hemostatically safe antithrombotic anticoagulant agent.

4. The method of claim 2 further comprising the step of using said compounds in combinations with other preventive and treatment therapies that target conditions characterized by vaso-occlusive diseases or conditions that are associated with increased risk of vaso-occlusive diseases.

5. The antithrombotic agents of claim 1 wherein said compounds that lack a paralyzing effect on hemostasis at any dose level specifically lack any effect against other proteins or enzymes of the extrinsic and/or common coagulation pathways, said other proteins or enzymes namely being coagulation factors I, II, V, VII, VIII, IX, X, and tissue factor.

6. The antithrombotic agents of claim 1 wherein:
said selective reduction of intravascular thrombin-generating thromboplastin antecedent activity at said optimal efficacious dose inhibits at least 20% and as much as 100% of the intravascular thrombin-generating thromboplastin antecedent activity; and,
said lacking a paralyzing effect on hemostasis equates to a maximum inhibition of less than 80% inhibition of hemostasis.

7. The antithrombotic agents of claim 1 wherein said compound is a pharmaceutically acceptable formulations with at least 10% better hemostatic safety than that of equiefficacious doses of other direct and indirect inhibitors of vascular occlusions.

8. The hemostatic safety of the effective dosage forms of the antithrombotic agents of claim 1, comprising the property of said compound equated to at least one of:
specific inhibition of activated thromboplastin antecedent, in vivo,
specific inhibition of thromboplastin antecedent activation, in vivo,
specific inhibition of thromboplastin antecedent production, in vivo,
enhancement of thromboplastin antecedent elimination, in vivo, and
enhancement of activated thromboplastin antecedent elimination, in vivo.

9. The antithrombotic agents of claim 1 wherein said agent is at least one compound selected from at least one of the categories consisting of:
a) small molecule enzyme inhibitors that interfere with or block the enzymatic activity of activated thromboplastin antecedent, when delivered to a human by pharmaceutically acceptable formulations and means;
b) neutralizing antibodies that inhibit activated thromboplastin antecedent activity or thromboplastin antecedent activation, when delivered to a human by pharmaceutically acceptable formulations and means;
c) polypeptides that interfere with activated thromboplastin antecedent activity or thromboplastin antecedent activation, when delivered to a human by pharmaceutically acceptable formulations and means;
d) peptidomimetics and small molecules that interfere with or block activated thromboplastin antecedent activity or thromboplastin antecedent activation, when delivered to a human by pharmaceutically acceptable formulations and means;
e) nucleic acid, DNA or RNA analogs that interfere with or block factor activated thromboplastin antecedent, thromboplastin antecedent activation, or thromboplastin antecedent production, or increase elimination of thromboplastin antecedent from the circulation when delivered to a human by pharmaceutically acceptable formulations and means; and,
f) thromboplastin antecedent, thrombin, or FXII analogs, related peptides and peptidomimetics that interfere with or block activated thromboplastin antecedent activity or thromboplastin antecedent activation, in vivo, when delivered to a human by pharmaceutically acceptable formulations and means.

10. The antithrombotic agents of claim 9 wherein said at least one compound is a dodecapeptide having the amino acid sequence of Glu-Glu-Val-Ala-Asn-Ala-Trp-Ser-Met-Ser-Pro-Ala, or Glu-Lys-Met-Glu-His-Gly-Ile-Trp-Asn-Arg-Thr-Ala.

11. The antithrombotic agents of claim 1 wherein said at least one compound is at least one of an immediate release pharmaceutical formulation, an extended release pharmaceutical formulation, administrable in a clinically efficacious dose to a patient by the enteral route and administrable in a clinically efficacious dose to a patient by the parenteral route.

12. A pharmaceutically acceptable preparation of an antithrombotic non-antihemostatic compound, comprising at least one active molecule, said at least one active molecule having at least the properties of:
rendering blood coagulation factor XI unable to participate in a reaction that is part of any thrombus producing pathway, and
at least one of exerting no effect on any other blood coagulation factor's participation in said any thrombus producing pathway and exerting an enhancing effect on at least one other blood coagulation factor's participation in said any thrombus producing pathway.

13. The antithrombotic of claim 12, wherein at least one said at least one active molecule is selected from the group consisting of:
a) a small molecule non-peptide enzyme inhibitor that specifically at least one of interferes with the enzymatic activity of FXIa, blocks the enzymatic activity of FXIa, interferes with the activation of FXI, blocks the activation of FXI, interferes with the production of FXI, and blocks the production of FXI;
b) an antibody or having a binding specificity for at least one of FXI, FXIa, and an FXI activating enzyme;
c) an antibody fragment having a binding specificity for at least one of FXI, FXIa, and an FXI activating enzyme;
d) an enzyme-antibody chimera having a binding specificity for at least one of FXI, FXIa, and an FXI activating enzyme;
e) an enzyme-antibody fragment chimera having a binding specificity for at least one of FXI, FXIa, and an FXI activating enzyme;
f) a polypeptides that at least one of interferes with FXIa activity and interferes with FXI activation;
g) a peptidomimetic that specifically at least one of interferes with the enzymatic activity of FXIa, blocks the enzymatic activity of FXIa, interferes with the activation of FXI, blocks the activation of FXI, interferes with the production of FXI, and blocks the production of FXI;
h) a small molecule peptide that specifically at least one of interferes with the enzymatic activity of FXIa, blocks the enzymatic activity of FXIa, interferes with the activation of FXI, blocks the activation of FXI, interferes with the production of FXI, and blocks the production of FXI;
i) one of a Nucleic acid, a DNA analog and an RNA analog, that at least one of directly interferes with the production of FXI, directly blocks the production of FXI, encodes a gene product that interferes with the production of FXI, encodes a gene product that blocks the production of FXI, encodes a gene product that interferes with the enzymatic activity of FXIa, encodes a gene product that blocks the enzymatic activity of FXIa, encodes a gene product that interferes with the activation of FXI, and encodes a gene product that blocks the activation of FXI;
j) a sequestration molecule that at least one of specifically isolates FXI, specifically aggregates FXI, specifically isolates FXIa, and specifically aggregates FXIa,
k) an inactive analog of one of FXI, thrombin, and FXII that specifically at least one of interferes with the enzymatic activity of FXIa, blocks the enzymatic activity of FXIa, interferes with the activation of FXI, blocks the activation of FXI, interferes with the production of FXI, and blocks the production of FXI; and
l) an inactive fragment of one of FXI, thrombin, and FXII that specifically at least one of interferes with the enzymatic activity of FXIa, blocks the enzymatic activity of FXIa, interferes with the activation of FXI, blocks the activation of FXI, interferes with the production of FXI, and blocks the production of FXI.

14. A method of treating or preventing a thrombosis or thrombin-dependent vaso-occlusive disease comprising the step of administering the antithrombotic of claim 12 to an individual.

15. The antithrombotic of claim 12, further comprising:
a device, said device having a surface adaptable to being exposed to circulating blood; and,
said at least one active molecule attached to said surface.

16. A method utilizing the antithrombotic of claim 12 for preventing a thromboses or thrombin-dependent vaso-occlusive diseases resulting from a medical/surgical procedure, comprising the steps of:
administering at least one sufficiently efficacious dose of said at least one active molecule of claim 1 sufficiently prior to, the initiation of said medical/surgical procedure to allow said at least one active molecule to significantly reduce a circulating concentrations of activatable FXI; and,
continuing to administer doses, of said at least one active molecule, sufficiently efficacious to maintain said significantly reduced circulating concentrations of activatable FXI, until sufficient time has elapsed that said thrombosis or thrombin-dependent vaso-occlusive diseases are no longer a threat.

17. A diagnostic assay utilizing the antithrombotic compounds of claim 12, comprising:
an agent to block the intrinsic pathway;
one of the said compounds of claim 1 that directly blocks the activation of FXI or the activity of FXIa;
a standard of at least one known concentration of activatable FXI, said standard having a similar composition to that of samples to be tested; and,
a diluent for diluting said samples, said agent to block, said one of the said compounds of claim 1, and said standard.

18. A method for diagnosing predisposition for developing a thromboses or thrombin-dependent vaso-occlusive disease, comprising the steps of:
determining a circulating FXI concentration for a patient;
applying a correlation algorithm to said circulating FXI concentration, said correlation algorithm being derived from a statistically determined population database of paired datum of a first datum for a survey subject's said circulating FXI concentration and a second datum for said survey subject's incidence of developing a thromboses or thrombin-dependent vaso-occlusive disease; and,
reading out the result from said algorithm of said patient's incidence of developing a thromboses or thrombin-dependent vaso-occlusive disease that correlates with said patient's said circulating FXI concentration
